(19) United States
(12) Patent Application Publication
(10) Pub. No.: US 2009/0098185 A1
(43) Pub. Date: Apr. 16, 2009

(54) Method for treating thrombotic disorders using sulfated polysaccharides

(75) Inventor: Kirk W. Johnson, Moraga, CA (US)

Correspondence Address: ROBINS & PASTERNAK LLP, 1731 EMBARCADERO ROAD, SUITE 230, PALO ALTO, CA, 94303, US

(73) Assignee: AVIGEN, INC., Alameda, CA (US)

(21) Appl. No.: 12/316,632

(22) Filed: Dec. 15, 2008

Related U.S. Application Data
(63) Continuation of application No. 11/789,447, filed on Apr. 24, 2007, now abandoned.
(60) Provisional application No. 60/797,079, filed on Apr. 27, 2006.

(51) Int. Cl. A61F 2/04; A61K 31/737

(52) U.S. Cl. 424/423; 514/54

(57) ABSTRACT

Methods for treating thrombotic disorders using sulfated polysaccharides such as fucoidans are disclosed.

### CROSS-REFERENCE TO RELATED APPLICATIONS

This application is a continuation application of U.S. application Ser. No. 11/789,447, filed Apr. 24, 2007, from which application priority is claimed pursuant to 35 USC §120, which application claims the benefit under 35 USC §119(e)(1) of U.S. Provisional Application Ser. No. 60/797,079, filed Apr. 27, 2006, which applications are incorporated herein by reference in their entireties.

### FIELD OF THE INVENTION

The invention relates to the treatment of thrombotic disorders, including deep vein thrombosis, pulmonary embolism, myocardial infarctions, prevention of stroke, and the treatment and prevention of blood clots, among others. In particular, this invention relates to the use of sulfated polysaccharides such as fucoidan to prevent coagulation.

### ABBREVIATIONS

The following abbreviations are used herein:

aPTT: activated partial thromboplastin time
dPT: dilute prothrombin time
PT: prothrombin time
DVT: deep-vein thrombosis
TF: tissue factor
FVIIa factor VIIa
FX factor X
FXa factor Xa
FVa factor Va
NSAID non-steroidal anti-inflammatory drug
TFPI tissue factor pathway inhibitor
NAPc2 nematode anticoagulant protein c2
HIT heparin-induced thrombocytopenia
HITTS heparin-induced thrombocytopenia thrombosis syndrome
TCT thrombin clotting time
ACT activated clotting time
INR international normalized ratio

### BACKGROUND OF THE INVENTION

Normal blood coagulation is a complex physiological and biochemical process that is regulated at several levels. The process of blood coagulation involves activation of a coagulation factor cascade leading to fibrin formation and platelet aggregation along with local vasoconstriction (reviewed by Davie et al., Biochemistry 30:10363, 1991). The clotting cascade is composed of an “extrinsic” pathway thought to be the primary means of normal coagulation initiation and an “intrinsic” pathway contributing to an expanded coagulation response. The normal response to a bleeding insult involves activation of the extrinsic pathway. Activation of the extrinsic pathway is initiated when blood comes in contact with tissue factor (TF), a cofactor for factor VII that becomes exposed or expressed on tissues following insult. TF forms a complex with FVII that facilitates the production of FVIIa. FVIIa then associates with TF to convert FX to the serine protease FXa, which is a critical component of the prothrombinase complex. The conversion of prothrombin to thrombin by the FXa/FVa/calcium/phospholipid complex stimulates the formation of fibrin and activation of platelets, all of which is essential to normal blood clotting. Normal hemostasis is further enhanced by intrinsic pathway factors IXa and VIIIa, which also convert FX to FXa. See also Weitz, J. I., et al., Chest, 126 (3), September 2004 (Suppl), 265S.

One important class of therapeutic agents are anticoagulants—agents that prevent the formation of blood clots. Anticoagulants are used to treat thrombotic disorders such as arterial and venous thrombosis, pulmonary embolism, myocardial infarction, and the prevention of stroke, among others. Anticoagulants are also used to prevent cardioembolic events in patients with atrial fibrillation or those possessing mechanical prosthetic heart valves.

Arterial and venous thrombosis are major causes of morbidity and mortality. Arterial thrombosis is the most common cause of myocardial infarction, stroke and limb gangrene, while venous thrombosis leads to pulmonary embolism (which can be fatal), and to postphlebitic syndrome.

Since arterial thrombi consist of platelet aggregates that are held together by small amounts of fibrin, strategies to inhibit aterial thrombogenesis are typically directed to drugs that block platelet function but also often include anticoagulant agents to prevent fibrin deposition. Venous thrombi are composed mainly of fibrin, thus, anticoagulants are the drugs of choice for their prevention and treatment.

Existing oral and parenteral anticoagulants are plagued by drawbacks. Warfarin (coumadin) is the primary oral anticoagulant used in clinical practice, and is a coumarin vitamin-K antagonist. Warfarin remains the only orally administered anticoagulant available for long-term prevention and treatment of venous thromboembolism. Unfortunately, warfarin possesses a narrow therapeutic index and requires frequent laboratory monitoring and dosage adjustment (J Ansell, Semin Vasc Surg 18:134, 2005). Moreover, a serious adverse side-effect of warfarin is the potential for excessive bleeding (e.g., with a cut, nosebleed, or menstruation). Further, warfarin can cause serious drug interactions, e.g., when co-administered with, e.g., certain antibiotics, acetaminophen, asprin, and NSAIDs (Ament, P. W., et al., American Family Physician, 61 (6), Mar. 15, 2000).

As a result of the limitations of warfarin, several new agents are being explored as potential anticoagulants. However, many of the potential anticoagulants under investigation, such as TFPI (tissue factor pathway inhibitor) or NAPc2 (nematode anticoagulant protein c2) are biologics, and are thus likely to be much more costly than existing anticoagulant therapies. Further, each is administered by a route that is less than desirable for the majority of the patient population—TFPI is administered intravenously while NAPc2 is administered subcutaneously.

Sulfated polysaccharides are a class of molecules characterized by a plethora of biological activities with often favorable tolerability profiles in animals and humans. These polyanionic molecules are often derived from plant and animal tissues and encompass a broad range of subclasses including heparins, glycosaminoglycans, fucoidans, carrageenans, pentosan polysulfates, and dermatan or dextran sulfates. Heparin-like sulfated polysaccharides exhibit differential anticoagulant activity mediated through antithrombin III and/or heparin cofactor II interactions (Toida T C, Linhardt, R J., Trends in Glycoscience and Glycotechnology 2003; 15:29-46).

While one such sulfated polysaccharide, oral heparin, has been considered for development as an anticoagulant (A Dunn, Idrugs, 3:817-824, 2000), heparin is inadequate because of its serious complications which include intraoperative and postoperative bleeding, osteoporosis, alopecia, heparin resistance, heparin rebound, heparin-induced thrombocytopenia (HIT), heparin-induced thrombocytopenia thrombosis syndrome (HITTS), and other disadvantages including multiple days for anticoagulation to attenuate after discontinuing the drug (Iqbal O, et al., Fareed J, Expert Opin Emerg Drugs 6:111-135, 2001; Roberts, H R, Anesthesiology 100:722-730, 2004). Heparin is conventionally administered parenterally, and possesses an oral uptake level of only about 1% (Fitton, J. H., Glycoscience, The Nutrition Science Site, modified Jan. 1, 2005).

In constrast to heparin, another sulfated polysaccharide, fucoidan, a sulfated polysaccharide isolated from sea algae, has been shown to regulate (i.e., promote) coagulation (U.S. Patent Publication No. 2005/0282771). Specifically, fucoidans, when administered at low concentrations in vitro, or low subcutaneous doses in vivo, provide improved (accelerated) clotting in hemophilic settings through extrinsic pathway activation (Liu, T., et al., and Johnson, K. W., Thrombosis and Haemostasis, 95:68-76, 2006).

In contrast to its function as a procoagulant and previous classification as a non-anticoagulant sulfated polysaccharide (NASP), the Applicants have discovered that when administered at higher doses, fucoidan possesses anticoagulant properties—surprisingly, even when administered orally.

In light of the problems associated with current anticoagulants, there clearly remains a need for new anti-clotting agents, and preferably agents that can overcome one or more of the above problems associated with currently available anticoagulant therapy. Thus, there exists a need for a pharmaceutical agent that is a safe, convenient, effective, and preferably cost-effective, anticoagulant. It is believed that the present invention meets this need.

### SUMMARY OF THE INVENTION

The present invention provides methods and compositions for treating various thrombotic disorders in a mammalian subject. In particular, the invention is directed to a method for treating a subject in need of reduced blood coagulation and comprises administering to such subject a composition comprising a therapeutically effective amount of one or more fucoidans. Preferably, the one or more fucoidans is administered orally.

Features of the fucoidan compositions include the following.

In one embodiment, the fucoidan component of the composition possesses from 5 to 25 percent by weight sulfur.

In yet another embodiment, the fucoidan is of algal origin.

In a preferred embodiment, the fucoidan is derived from the genus Fucus or Laminaria. Illustrative fucoidans are those derived from Fucus vesiculosis or from Laminaria japonica.

In one embodiment, the method of administering is effective to produce a greater than 50% prolongation in blood coagulation time, typically measured as prolongation in clotting time using a suitable clot-based assay such as the aPTT assay.

The fucoidan compositions of the invention are useful in treating or preventing conditions including venous thromboembolism, deep vein thrombosis, pulmonary embolism, coronary artery disease (coronary thrombosis, coronary angioplasty), unstable angina or acute myocardial infarction, coronary thrombolysis, atrial fibrillation, stroke, disseminated intravascular coagulation, and procoagulation or thrombosis induced by Factor VIIa treatment.

The compositions of the invention may also be administered to prevent thrombosis in patients undergoing general surgery, those undergoing major orthopedic procedures, suffering hip fracture, or undergoing neurosurgery. The compositions of the invention may also be administered concurrent with the use of a compression stocking, e.g., for clot prevention in the lower extremities.

Also provided herein is a method for reversing the effects of use of a procoagulant in a subject, the method comprising administering a therapeutically effective amount of a fucoidan composition as described herein to the subject.

These and other embodiments of the subject invention will be readily apparent to those of skill in the art in view of the present disclosure

### BRIEF DESCRIPTION OF THE FIGURES

FIG. 1 is a plot demonstrating the plasma clotting time (in seconds) over a timecourse of 7 hours in normal beagle dogs following oral administration of 20 mg/kg fucoidans as described in Example 1. Plasma clotting times were determined using an aPTT assay.

### DETAILED DESCRIPTION OF THE INVENTION

The practice of the present invention will employ, unless otherwise indicated, conventional methods of pharmacology, chemistry, biochemistry, coagulation, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a fucoidan” includes a mixture of two or more particular fucoidans, and the like.

An “anticoagulant” as used herein refers to any agent capable of preventing or slowing clot formation.

The term “polysaccharide,” as used herein, refers to a polymer comprising a plurality (i.e., two or more) of covalently linked saccharide residues. Linkages may be natural or unnatural. Natural linkages include, for example, glycosidic bonds, while unnatural linkages may include, for example, ester, amide, or oxime linking moieties. Polysaccharides may have any of a wide range of average molecular weight (MW) values, but generally are of at least about 100 daltons. For example, the polysaccharides can have molecular weights of at least about 500, 1000, 2000, 4000, 6000, 8000, 10,000, 20,000, 30,000, 50,000, 100,000, 500,000 daltons or even higher. Polysaccharides may have straight chain or branched structures. Polysaccharides may include fragments of polysaccharides generated by degradation (e.g., hydrolysis) of larger polysaccharides. Degradation can be achieved by any of a variety of means known to those skilled in the art including treatment of polysaccharides with acid, base, heat, or enzymes to yield degraded polysaccharides. Polysaccharides may be chemically altered and may have modifications, including but not limited to, sulfation, polysulfation, esterification, and methylation.

The term “derived from” is used herein to identify the original source of a molecule but is not meant to limit the method by which the molecule is made which can be, for example, by chemical synthesis or recombinant means.

By “derivative” is intended any suitable modification of the parent molecule of interest or of an analog thereof, such as sulfation, acetylation, glycosylation, phosphorylation, polymer conjugation (such as with polyethylene glycol), or other addition of foreign moieties, so long as the desired biological activity (e.g., anticoagulant activity) of the parent molecule is retained to at least a significant degree (e.g., such that at least 20% of the desired biological activity of the parent molecule is retained). For example, polysaccharides may be derivatized with one or more organic or inorganic groups. Examples include polysaccharides substituted in at least one hydroxyl group with another moiety (e.g., a sulfate, carboxyl, phosphate, amino, nitrile, halo, silyl, amido, acyl, aliphatic, aromatic, or a saccharide group), or where a ring oxygen has been replaced by sulfur, nitrogen, a methylene group, etc. Polysaccharides may be chemically altered, for example, to improve anticoagulant function. Such modifications may include, but are not limited to, sulfation, polysulfation, esterification, and methylation. Methods for making analogs and derivatives are generally available in the art. See for example, “Chemistry of Polysaccharides”, Ed. G. E. Zaikov, 2005, VSP Publishers, the Netherlands; “Industrial Polysaccharides”, Proceedings of the Symposium of the Applications and Modifications of Industrial Polysaccharides”, held during the 193^{rd} ACS National Meeting, Denver Colo., USA, Apr. 5-10, 1987, Ed. M. Yalpani, Elsevier.

By “fragment” is intended a molecule consisting of only a part of the intact full-length sequence and structure. A fragment of a polysaccharide may be generated by degradation (e.g., hydrolysis) of a larger polysaccharide. Active fragments of a polysaccharide will generally include at least about 2-20 saccharide units of the full-length polysaccharide, preferably at least about 5-10 saccharide units of the full-length molecule, or any integer between, 2 saccharide units and the full-length molecule, provided that the fragment in question retains biological activity, such as anticoagulant activity.

“Purified” generally refers to isolation of a substance (e.g., sulfated fucan) such that the substance comprises the majority weight percent of the overall sample. Typically in such a sample, a purified component comprises greater than 50% by weight, preferably 80%-85%, and even more preferably 90-95% of the sample. Techniques for purifying polysaccharides are well-known in the art and include, for example, ion-exchange chromatography, affinity chromatography and sedimentation according to density.

By “isolated” is meant, when referring to a polysaccharide or polypeptide, that the indicated molecule is separate and discrete from the whole organism with which the molecule is found in nature or is present in the substantial absence of other biological macromolecules of the same type.

“Substantially” or “essentially” means nearly totally or completely, for instance, 95% or greater of some given quantity.

“Optional” or “optionally” means that the subsequently described circumstance may or may not occur, so that the description includes instances where the circumstance occurs and instances where it does not.

A subject in need of “reduced blood coagulation” is one that has been identified as being at risk for clot formation (e.g., possesses an artificial heart valve, has had a heart attack or stroke, has had or is at risk of deep vein thrombosis, possesses atrial fibrillation, has developed a blood clot for no apparent reason, is undergoing orthopaedic surgery, has angina) or the like, is undergoing Factor VIIa treatment, or has been assessed, e.g., as a result of a clinical blood clotting test such as aPPT or dPT or the like, as having blood that clots more readily than normal.

Molecular weight, in the context of a fucoidan of the invention, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques.

The terms “pharmacologically effective amount” or “therapeutically effective amount” of a composition or agent such as fucoidan, refer to a nontoxic but sufficient amount of the composition or agent to provide the desired response, e.g., extended blood clotting times (or reduced blood coagulation). The exact amount required will vary from subject to subject, depending on the species, age, and general condition of the subject, the severity of the condition being treated, the particular drug form or combination of drugs employed, mode of administration, and the like. An appropriate “effective” amount in any individual case may be determined by one of ordinary skill in the art using routine experimentation, based upon the information provided herein.

The terms “subject”, “individual” or “patient” are used interchangeably herein and refer to a vertebrate, preferably a mammal. Mammals include, but are not limited to, murines, rodents, simians, humans, farm animals, sport animals and pets.

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species.

The term “about”, particularly in reference to a given quantity, is meant to encompass deviations of plus or minus five percent.

Recommended dosage amounts for a given therapeutic agent, when expressed in mg/kg, refer to mg of therapeutic agent per kg of body mass of the subject.

### DETAILS OF THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

General Overview

Currently, there are very few oral anticoagulant therapeutic options. Warfarin is the most common and, while it is effective, it is difficult to safely titrate due to its narrow therapeutic index. Moreover, its duration of anticoagulant action is long-lasting (on the order of several days), which can further complicate its safe use (HR Roberts Anesthesiology 100:722-730, 2004). As a result, warfarin is vastly underutilized by clinicians (Wittkowsky, A. K., The American Journal of Managed Care, October 2004, Supp., S297-306), understating the need for new, oral anticoagulants.

Even though fucoidan is comprised of large, heterogeneous sulfated polysaccharides, and is not generally thought to be orally bioavailable, it has been demonstrated by the Applicants that relatively low doses of fucoidan administered orally yield a detectable anticoagulant response. In particular, in an investigation of the use of oral fucoidan as a useful, effective anticoagulant, the Applicants have demonstrated that oral fucoidan possesses anticoagulant efficacy, and should be safer than warfarin based on proposed mechanisms of i) anticoagulation at higher concentrations via ATIII and/or HCII inhibition (Toida T C, Linhardt, R J., Trends in Glycoscience and Glycotechnology 2003; 15:29-46), ii) low concentration enhancement of extrinsic pathway activation (Liu, T., et al., and Johnson, K. W. Thrombosis and Haemostasis, 95:68-76, 2006), iii) a duration of action which is hours (see Examples herein) versus days for warfarin, iv) improved safety margin relative to warfarin, and v) more rapid initial onset of anticoagulation (i.e., faster “on”, faster “off” than warfarin).

Fucoidans as Oral Anticoagulants

The present invention is based on the discovery that fucoidans such as those derived from the genus Fucus or Laminaria, among others, can be administered orally and are effective as anticoagulating agents in the treatment of patients in need of reduced blood coagulation.

The approach herein is based on the use of sulfated polysaccharides, such as heparin-like sulfated polysaccharides, to reduce the tendency for or prevent blood clots. Selected sulfated polysaccharides described herein have been found to possess anticoagulant activity when administered orally, at concentrations significantly higher than the concentration at which they exhibit pro-coagulant activity. Moreover, their onset of action is quite rapid, e.g., typically on the order of about an hour when administered orally to beagle dogs, with a duration of anticoagulant activity of several (e.g., eight or so) hours. This presents a notable advantage over other oral agents such as warfarin, whose duration of action is on the order of days—which presents problems with the tendency for excessive bleeding and the need for frequent patient monitoring.

As can be seen in Example 1, fucoidan administered to healthy beagle dogs exhibited dose-related anticoagulant activity in each of the three dogs treated, as exhibited by significant (>50%) increases in the aPTT, the most commonly utilized clinical measurement for blood coagulation. Specifically, oral administration of a single 20 mg/kg dose of an illustrative fucoidan composition to each of three healthy beagle dogs was effective to increase plasma clotting times in each of the three dogs by approximately five to over nine times the pre-dose values within times ranging from about 1 hour to 7 hours post administration. For dogs #1 and #2, significant increases (i.e., from approximately six times to over nine times initial pre-dose values) in plasma clotting times were observed within about 0.5 hours to 1 hour post-administration. Moreover, for dogs #1 and #2, plasma clotting times returned to essentially pre-dose values within several hours post-administration, indicating a potential for a lessened haemorrhagic effect when compared to a drug such as warfarin.

Thus, the invention relates to the use of fucoidans in subjects in need of reduced blood coagulation, including those suffering from a condition selected from deep vein thrombosis, pulmonary embolism, myocardial infarction, disseminated intravascular coagulation, unstable angina, and procoagulation or thrombosis induced by Factor VIIa treatment, among others.

Fucoidans

Fucoidans are naturally-occurring components of certain edible seaweeds and echinoderms. More particularly, they are complex sulfated polysaccarides constituted mainly of sulfated L-fucose and are derived from kelp (marine brown algae) and echinodemms such as sea urchins and sea cucumbers (Carbohydrate-based Drug Discovery, Vol. 1, Wong, Chi-Huey (Ed.), Chapter 15, p. 407-433, Wiley-VCH, 2003). The term, “fucoidan” typically refers to a diverse group of moieties of low sulfate polymers rather than a single chemical entity. Fucoidans are primarily composed of α(1-3) linked units of 4-sulfo-L-fucose with branching or a second sulfo group at position 3 (Wong, ibid). Fucoidan from various species of brown algae and echinoderm differ in the amount of fucose in their backbone, the degree and pattern of sulphation, structure (linear versus branching), and proportions of individual saccharides and uronic acid. Preferred fucoidans for use in the invention are those derived from brown algae.

Fucoidans for use in the present invention may be extracted, further purified and/or modified from natural sources (e.g. brown algae) or may be synthesized de novo. Fucoidans can be isolated from algae by hot water (Percival and Ross, J. Chem. Soc., 1950, 717-720), by acid or ethanol extraction, or by enzymatic digestion, followed by isolation from aqueous solution by precipitation (e.g., by addition of organic solvents) or ultrafiltered (see, e.g., Black, WAP et al., IV J Sci Food Agric. 1952; 3: 122-129). Fucoidan is also commercially available from various sources such as Sigma (St. Louis, Mo.), NaturoDoc LLC (Kingman, Ariz.), Marinova (Tazmania, Australia) and Natureza Co., Ltd., (Kawaguchi, JP). Preferred fucoidans for use in the present invention are fucoidans derived from the genus Fucus or from the genus Laminaria, although fucoidans from other genuses are also suitable. Such fucoidans are referred to herein as Fucus fucoidans or Laminaria fucoidans. Additional fucoidans for use in the invention include fucoidans derived from Cladosiphon, Namacystus, Undaria, Chordaria, Sargassum, Leathesia, Desmarestia, Dictyosiphon, Dictyota, Padina, Spatoglossum, Adenocystis, Pylayella, Ascophyllum, Bifurcaria, Himanthalia, Hizikia, Pelvetia, Alaria, Arthrothamnus, Chorda, Ecklonia, Eisenia, Macrocystis, Nereocystis, Petalonia, Scytosiphon, and Saundersella, among others. Particularly preferred fucoidans are derived from Fucus vesiculosis or from Laminaria japonica.

Fucoidans for use in the methods and compositions herein are typically although not necessary heterogeneous mixtures of fucoidans varying in molecular weight, fucose content, uronic acid content, and sulfate content.

Fucoidan may range in average molecular weight from about 200 daltons to about 500,000 daltons, preferably from about 1,000 daltons to about 300,000 daltons. Fucoidan for use in the present invention includes low molecular weight fucoidan (having a weight average molecular mass in the range from about 500 to 8,000 daltons), middle molecular weight fucoidan (having a weight average molecular mass ranging from about greater than 8,000 daltons to about 15,000 daltons) and high molecular weight fucoidan (having a weight average molecular mass ranging from greater than 15,000 daltons to about 300,000 daltons). Molecular weights of fucoidan can be determined, e.g., using gel permeation chromatography or high-performance steric chromatography (HPSEC).

Differing molecular weight fractions of fucoidan may be separated by capillary electrophoresis. Alternatively, different molecular weight fractions may be prepared by acid-hydrolysis or radical depolymerization of high molecular weight fucoidan. The molecular weight ranges of the resulting products may be adjusted based upon the stringency of the hydrolysis or depolymerization conditions employed. Fractions may then be further purified using ion exchange chromatography. For instance, to obtain middle and low molecular weight fractions of fucoidan, high molecular weight fucoidari is hydrolyzed using an acid such as HCl (or any other suitable acid) at concentrations ranging from 0.02 to 1.5 M and at temperatures ranging from 25° C. to 80° C. Hydrolysis reaction times will typically range from 15 minutes to several hours. The resulting hydrolyzed reaction mixture is then neutralized by addition of base, e.g., sodium hydroxide. Salts are then removed, e.g., by electrodialysis, and the hydrolysis products are analyzed to determine weight average molecular weight, fucose content, uronic acid content, and sulfate content, using conventional analytical techniques for carbohydrate analysis. Alternatively, enzymatic methods may be employed to degrade fucoidans using, e.g., glycosidases such as fucan sulfate hydrolase (fucoidanase EC 3.2.1.44) and α-L-fucosidase EC 3.2.1.51. Fucoidans for use in the invention may be heterogeneous or homogeneous, depending upon the degree of separation employed. See, e.g., Nardella, A., et al., Carbohydr Res 1996; 289:201-208; Deux, J. F., et al., Arteriosclerosis, Thrombosis, and Vascular Biology. 2002; 22:1604; Zemani, F., et al., Biochemical Pharmacology, October 2005; 70(8): 1167-1175.

Fucoidans for use in the invention will typically possess from about 5 to 25% by weight sulfur, preferably from 8-25% by weight sulfur. Fucoidans possessing greater than about 5% by weight sulfur, or even greater than 10% by weight sulfur, are particularly preferred.

In principle, any free hydroxyl group on a monosaccharide component of a fucose polysaccharide can be modified by sulfation to produce a sulfated higher saccharide (di-, tri, oligo-, or poly-) for use in the practice of the invention. Sulfation is typically carried out using sulfur trioxide complexes with pyridine or triethylamine, or with stannous complexes. (Calvo-Asin, J. A., et al., J. Chem. Soc, Perkin Trans 1, 1997, 1079). For example, one or more disaccharides related to fucoidan may be stereoselectively synthesized by formation of an α-fucose bond between two suitable monosaccharide precursors using, e.g., 3,4-di-O-acetylated trichloroacetoimidate as a fucosyl donor (See e.g., Zlotina, N. S., et al., L. Hirszfeld Institute of Immunology and Experimental Therapy, Polish Academy of Sciences, 1^{st} Baltic Meeting on Bacterial Carbohydrates, Oct. 6-9, 2004, Wroclaw, Poland).

Clotting Assays

The ability of a fucoidan to prevent clotting is readily determined using various well-established coagulation assays including clot-based tests, chromogenic or color assays, direct chemical measurement, and ELISA, although clot-based tests are the most common. These include PT (prothrombin time), aPTT (activated partial thromoplastin time), ACT (activated clotting time), and TCT (thrombin clotting time). See, e.g., Bates, S. M., et al, Circulation, 2005; 112: e53-60; PDR Staff. Physicians' Desk Reference. 2004, Anderson et al. (1976) Thromb. Res. 9:575-580; Nordfang et al. (1991) Thromb Haemost. 66:464-467; Welsch et al. (1991) Thrombosis Research 64:213-222; Broze et al. (2001) Thromb Haemost 85:747-748; Scallan et al. (2003) Blood. 102:2031-2037; Pijnappels et al. (1986) Thromb. Haemost. 55:70-73; and Giles et al. (1982) Blood 60:727-730.

Prothrombin Time (ET). This test is performed by adding a thromboplastin reagent that contains tissue factor (which can be recombinant in origin or derived from an extract of brain, lung, or placenta) and calcium to plasma and measuring the clotting time. The prothrombin time (PT) varies with reagent and coagulometer but typically ranges between 10 and 14 seconds (White, GC II, et al., Approach to the Bleeding Patient. InL Colman, R W, et al., eds. Hemostasis and Thrombosis: Basic Principles and Clinical Practice. 3^{rd} ed. Philadelphia, Pa.: JB Lippincott Co., 1994: 1134-1147). The PT is prolonged with deficiencies of factors VII, X, and V, prothrombin, or fibrinogen and by antibodies directed against these factors. This test also is abnormal in patients with inhibitors of the fibrinogen-to-fibrin conversion reaction, including high doses of heparin and the presence of fibrin degradation products. Typically, PT reagents contain excess phospholipid so that nonspecific inhibitors (i.e., lupus anticoagulants), which react with anionic phospholipids, do not prolong the clotting time.

Activated partial thromoplastin time (aPTT). The aPTT is performed by first adding a surface activator (eg, kaolin, celite, ellagic acid, or silica) and diluted phospholipid (eg, cephalin) to citrated plasma. The phospholipid in this assay is called partial thromboplastin because tissue factor is absent. After incubation to allow optimal activation of contact factors (factor XII, factor XI, prekallikrein, and high-molecular-weight kininogen), calcium is then added, and the clotting time is measured. Although the clotting time varies according to the reagent and coagulometer used, the aPTT typically ranges between 22 and 40 seconds (Bates, et al., ibid). The aPTT may be prolonged with deficiencies of contact factors; factors IX, VIII, X, or V; prothrombin; or fibrinogen. Specific factor inhibitors, as well as nonspecific inhibitors, may also prolong the aPTT. Fibrin degradation products and anticoagulants also prolong the aPTT.

Thrombin Clotting Time (TCT). TCT is performed by adding excess thrombin to plasma. The TCT is prolonged in patients with low fibrinogen levels or dysfibrinogenemia and in those with elevated fibrin degradation product levels. These abnormalities are commonly seen with disseminated intravascular coagulation.

Activated clotting time (ACT). The ACT is a point-of-care whole-blood clotting test typically used to monitor high-dose heparin therapy or treatment with bivalirudin. Typically, whole blood is collected into a tube or cartridge containing a coagulation activator (e.g., celite, kaolin, or glass particles) and a magnetic stir bar, and the time taken for the blood to clot is then measured. The reference value for the ACT ranges between 70 and 180 seconds. The desirable range for anticoagulation depends on the indication and the test method used.

Such clotting assays, or other suitable assays as described briefly above, may be performed in the presence of one or more fucoidans, and optionally, one or more blood factors, anticoagulants, or other reagents, and the values compared both before and after treatment to determine the extent of prolongation of blood coagulation times.

The fucoidans used in the methods and compositions of the present invention are such that any procoagulant activity that they may exhibit only appears at concentrations significantly below the concentration at which they exhibit anticoagulant activity. Such procoagulant activity is additionally typically exhibited following several days of administration rather than following a single dose as is the case in the present invention. The ratio of the concentration at which undesired procoagulant properties occur to the concentration at which desired anticoagulant activities occur is referred to as the therapeutic index for the subject therapeutic fucoidan.

The fucoidan composition of the present invention is typically administered to a subject in an amount sufficient to maintain the ratio of the patient's aPTT to a mean control aPTT within a defined range of approximately 1.5 to 2.5, referred to as the therapeutic range.

Pharmaceutical Compositions and Dosage Forms

Optionally, the fucoidan compositions of the invention may further comprise one or more pharmaceutically acceptable excipients to provide a pharmaceutical composition. Preferably, the fucoidan comprising the composition is essentially free of algal components other than the fucoidan(s) itself. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the fucoidan or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite; sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the fucoidan in the composition will vary depending on a number of factors, but will optimally be a therapeutically effective dose when the composition is in a unit dosage form (e.g., tablet, capsule, or the like) or container (e.g., a vial). A therapeutically effective dose can be determined experimentally by repeated administration of increasing amounts of the composition in order to determine the optimal amount effective to produce a clinically desired endpoint—in this case, the prevention of blood clots, as measured by prolonged blood clotting times.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters of the composition, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations including those that are suited for oral administration as well as parenteral formations. One particularly preferred formulation is one suited for oral administration. Oral dosage forms include powders, tablets, lozenges, capsules, syrups, solutions (liquids), oral suspensions, emulsions, granules, and pellets. Alternative formulations include aerosols, transdermal patches, gels, creams, ointments, sprays, suppositories, powders or lyophilates that can be reconstituted, as well as liquids. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof.

Topical formulations may additionally include a compound that enhances absorption or penetration of the ingredients through the skin or other affected areas, such as dimethylsulfoxidem bisabolol, oleic acid, isopropyl myristate, and D-limonene, to name a few.

Formulations suitable for parenteral administration include aqueous and non-aqueous isotonic sterile solutions suitable for injection, as well as aqueous and non-aqueous sterile suspensions. Parenteral formulations are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A fucoidan formulation of the invention may also be in the form of a sustained release formulation, such that the fucoidan and optionally other drug components are released or absorbed slowly over time, when compared to a non-sustained release formulation. Sustained release formulations may employ pro-drug forms of the active agent, delayed-release drug delivery systems such as liposomes or polymer matrices, hydrogels, or covalent attachment of a polymer such as polyethylene glycol.

In addition to the ingredients particularly mentioned above, the formulations of the invention may optionally include other agents conventional in the pharmaceutical arts and particular type of formulation being employed, for example, for oral administration forms, the composition for oral administration may also include additional agents as sweeteners, thickeners or flavoring agents.

The method of the present invention is also useful in veterinary applications.

The invention further encompasses a kit, e.g., for treatment of a mammalian subject in need of reduced blood coagulation. The kit comprises fucoidan, in packaged form, preferably for oral administration, accompanied by instructions for use. For example, the kit includes instructions for administering a recommended dosage of fucoidan to a patient in need of reduced blood coagulation—i.e., to prevent blood clot formation. The instructions will preferably recommend orally administering 1 mg/kg to 50 mg/kg of the fucoidan component of the composition daily for effecting reduced blood coagulation. The fucoidan may be packaged in any manner suitable for administration, so long as the packaging, when considered along with the instructions for administration, clearly indicates the manner in which the drug (i.e., fucoidan) component is to be administered. For example, when fucoidan is in the form of a capsule, then the kit may comprise a sealed container of capsules, blister strips containing the capsules, or the like. The packaging may be in any form commonly employed for the packaging of pharmaceuticals, and may utilize any of a number of features such as different colors, wrapping, tamper-resistant packaging, blister paks or strips, dessicants, and the like.

Administration

At least one therapeutically effective cycle of treatment with a fucoidan composition as provided herein will be administered to the subject. By “therapeutically effective cycle of treatment” is intended a cycle of treatment that when administered, brings about a positive therapeutic response with respect to treatment of an individual for a thrombotic disorder. Of particular interest is a cycle of treatment with a fucoidan composition that reduces the tendency of the blood to coagulate (i.e., prevents the formation of blood clots). Such positive therapeutic response can be measured by one or more of the coagulation assays described above to provide a desired response. By “positive therapeutic response” is intended that the individual undergoing treatment according to the invention exhibits an improvement in one or more symptoms of the subject thrombotic condition or disorder, including such improvements as prolonged blood clotting times and improved bleeding.

For instance, since commercially available thromboplastins vary in their tissue factor source and method of preparation, reported PT times can vary depending upon the reagents employed. A parameter called the INR was developed by the World Health Organization (WHO) to correct for differences in PT results. The INR is defined as the patient PT divided by the mean normal PT value, which can be calculated using an international sensitivity index which describes the responsiveness of each thromboplastin reagent compared to a standard (Bates, S. M., et al., ibid). Optimal therapeutic ranges for the INR in various indications are as follows and can be used to monitor the course of treatment when administering the fucoidan compositions of the invention: venous thromboembolism, prevention and treatment (2.0-3.0), atrial fibrillation (2.0-3.0), valvular heart disease (2.0-3.0), heart valves—tissue valves, mechanical valves, bileaflet aortic position (2.0-3.0), heart valves, mechanical, high risk valve (2.5-3.5), acute myocardial infarction-prevention of embolism (2.0-3.0), acute myocardial infarction—prevention of reinfarction (3.5-4.5). In one particular embodiment of the method, a fucoidan composition of the invention is administered in an amount effective to produce a greater than 50% improvement in the subject's aPTT. Preferably, the methods and compositions of the invention are effective to produce at least a two-fold or greater prolongation in the subject's aPTT. The methods and compositions of the invention are ideally effective to provide an INR range of about 1-10.

In certain embodiments, multiple therapeutically effective doses of a fucoidan compositions of the invention optionally comprising one or more other therapeutic agents, such as factor VIIa, warfarin, thrombin inhibitors, low molecular weight heparins, and heparin, or other medications, will be administered. The compositions of the present invention are typically, although not necessarily, administered orally, although injection (subcutaneously, intravenously or intramuscularly), infusion, and local administration are also contemplated. Additional modes of administration are also contemplated, such as pulmonary, rectal, transdermal, transmucosal, intrathecal, pericardial, intra-arterial, intracerebral, intraocular, intraperitoneal, and so forth.

In a particular embodiment, a fucoidan composition of the invention is used for localized delivery, for example, for the direct treatment of a blood clot. For example, the fucoidan may be administered by injection at the site of the clot. The particular preparation and appropriate method of administration are chosen based on the particular condition of the subject.

In another embodiment, the pharmaceutical compositions of the invention are administered prophylactically, e.g. before or after a planned surgery. Such prophylactic uses will be of particular value for subjects with known pre-existing blood coagulation disorders.

In another embodiment of the invention, the pharmaceutical compositions comprising fucoidan and/or other agents, are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The method of administering is used to treat any condition associated with the need for anticoagulant therapy. Such conditions include thrombotic conditions such as deep vein thrombosis, pulmonary embolism, myocardial infarction, disseminated intravascular coagulation, unstable angina, and procoagulation or thrombosis induced by Factor VIIa treatment.

Those of ordinary skill in the art will appreciate which conditions, such as those described above, a specific fucoidan composition of the invention can effectively treat. The actual dose to be administered will vary depending upon the age, weight, and general condition of the subject as well as the severity of the condition being treated, the judgement of the health care professional, and particular fucoidan(s) being administered. Therapeutically effective amounts can be determined by those skilled in the art, and will be adjusted to the particular requirements of each particular case.

Generally, a therapeutically effective amount will range from about 1 mg/kg to about 50 mg/kg of fucoidan daily, more preferably from about 2 mg/kg to 40 mg/kg daily, even more preferably from about 5 mg/kg to 30 mg/kg daily. Preferably, such doses are in the range of 0.25-12 mg/kg four times a day (QID), 0.5-10 mg/kg QID, 1.25-7.5 mg/kg QID, 0.3-17 mg/kg three times a day (TID), 0.6-13 mg/kg TID, 1.5-10 mg/kg TID, 0.05-25 mg/kg twice daily (BID), 1-20 mg/kg BID, or 2.5-15 mg/kg BID. Overall daily dosages will generally range from about 50 mg fucoidan to about 4500 mg fucoidan, depending upon several factors such as the body mass of the subject, age of the subject, the condition being treated, the potency of the specific fucoidan(s) contained in the composition, the magnitude or anticoagulant effect desired and the particular route of administration.

The fucoidan compositions described herein can be administered alone or in combination, or with other therapeutic agents, to treat a particular condition or disease according to a variety of dosing schedules depending on the judgement of the clinician, needs of the patient, and so forth. The specific dosing schedule will be readily determined by those of ordinary skill in the art or can be determined experimentally using routine methods. Exemplary dosing schedules include, without limitation, administration of the subject fucoidan composition five times a day, four times a day, three times a day, twice daily, once daily, three times weekly, twice weekly, once weekly, twice monthly, once monthly, and any combination thereof. Most preferred are compositions requiring dosing no more than once a day.

Applications

In one aspect, the fucoidan compositions of the invention are used for treating thrombotic disorders. The compositions of the invention are administered to a subject in need of reduced blood coagulation. Conditions to be treated or prevented include venous thromboembolism, deep vein thrombosis, pulmonary embolism, coronary artery disease (coronary thrombosis, coronary angioplasty), unstable angina or acute myocardial infarction, coronary thrombolysis, atrial fibrillation, stroke, disseminated intravascular coagulation, and procoagulation or thrombosis induced by Factor VIIa treatment. The compositions of the invention may also be administered to prevent thrombosis in patients undergoing general surgery, those undergoing major orthopedic procedures, suffering hip fracture, or undergoing neurosurgery. The compositions of the invention may also, be administered concurrent with the use of a compression stocking, e.g., for clot prevention in the lower extremities.

The invention also provides a method for reversing the effects of use of a procoagulant in a subject, the method comprising administering a therapeutically effective amount of a fucoidan composition to the subject.

In certain embodiments, the subject may have been previously treated with a procoagulant including, but not limited to, thrombin; an activator of the intrinsic coagulation pathway, including factor Xa, factor IXa, factor XIa, factor XIIa, and VIIIa, prekallekrein, and high-molecular weight kininogen; or an activator of the extrinsic coagulation pathway, including tissue factor, factor VIIa, factor Va, and factor Xa.

Additionally, the fucoidan compositions of the invention may be used as a coating for stents and other blood-contacting medical devices such as membrane oxygenators for cardiopulmonary bypass or mechanical heart valves to reduce local coagulation from blood-instrument interactions. Fucoidan compositions of the invention may be applied directly as a coating to the blood-contacting device, in one or in multiple layers. Such devices are referred to as “fucoidanized”. Alternatively, the fucoidan composition may be applied in combination with one or more additional hemocompatible polymers such as human serum albumin, phosphorylcholine, poly(D,L-lactide-co-glycolide), polyethylene glycol, polypropylene-oxide-polyethylene glycol block copolymers (PPO-PEG), phosphazene polymers, and the like. The fucoidan may also be releasably immobilized on the surface of the device by covalent coupling to a polymer coating using a coupling agent such as glutaraldehye, 1,1-carbonyldiimidazole, or any other suitable coupling agent. Such coupling agents are well-known in the art. See, e.g., Wong, S. S., Chemistry of Protein Conjugation and Cross-Linking, CRC Press, 1991.

It is to be understood that while the invention has been described in conjunction with preferred specific embodiments, the foregoing description as well as the example that follows are intended to illustrate and not limit the scope of the invention. Other aspects, advantages and modifications within the scope of the invention will be apparent to those skilled in the art to which the invention pertains.

### EXAMPLES

The practice of the invention will employ, unless otherwise indicated, conventional techniques of pharmaceutical formulation, separations, pharmacology, and the like, which are within the skill of the art. Such techniques are fully explained in the literature. See, for example, Handbook of Pharmaceutical Manufacturing Formulations, S. K. Niazi (ed.), CRC Press, 2004; Goodman & Gilman, The Pharmacological Basis of Therapeutics, 9^{th} Edition, Hardman, J. G., Gilman, A. G., Limbird, L. E. (eds.), McGraw-Hill, New York, 1995; Basic and Clinical Pharmacology, 18^{th} Edition, Katzung, B. G. (ed.), Appleton & Lange, Norwalk, Conn., 2001.

In the following examples, efforts have been made to ensure accuracy with respect to numbers used (e.g., amounts, temperatures, etc.) but some experimental error and deviation should be accounted for. Each of the following examples is considered to be instructive to one of ordinary skill in the art for carrying out one or more of the embodiments described herein.

### Material and Methods

### Reagents

Fucoidan was purchased from Sigma (St. Louis, Mo.).

### Animals

Healthy beagle dogs were obtained from Covance Laboratories colony. All animal procedures were performed according to “Guide for the Care and Use of Laboratory Animals” (National Research Council. Guide for the care and use of laboratory animals. Washington, D.C.: National Academy Press; 1996) and all procedures were reviewed and approved by an Institutional Animal Care and Use Committee.

### Clotting Assays

Activated Partial Thromboplastin Time (aPTT) Assay

The aPTT assays were performed as described in Liu, T., et al., and Johnson, K. W., Thrombosis and Haemostasis, 95: 68-76, 2006.

Dilute Prothrombin Time (dPT) Assay

The dPT assays were performed as described in Liu, T., et al., and Johnson, K. W., Thrombosis and Haemostasis, 95: 68-76, 2006.

### Example 1

### In-Vivo Evaluation of Anticoagulant Activity of Fucoidans Upon Administration to Healthy Beagle Dogs

The following study was performed to examine the oral anticoagulant activity of an exemplary fucoidan composition in beagle dogs using clinically-based clotting assays. Both the aPTT and dPT assays were conducted on plasma samples withdrawn at multiple time points during the study.

Three normal beagle dogs were used for the study. Each animal was administered one single fucoidan oral dose (20 mg/kg or 5 mg/kg) delivered as a powder in gelatin capsules (size “0”), with a one week washout period between dosings.

Clinical observations were performed up to 7 hours post-fucoidan dosing. Plasma samples were collected (titrated whole blood, plasma isolation) at pre-dose, 15 min, 30 min, 1 hr, 2 hr, 4 hr, and 7 hr post-fucoidan administration. Plasma samples were stored at −20° C. prior to testing.

The assay results (in duplicate) are provided for each of dogs #1-3 below.

TABLE 1
aPTT Assay Results for 20 mg/kg Oral Dosage Amount

| | Dog #1 | | Dog #2 | | Dog #3 | |
|---|---|---|---|---|---|---|
| | CTAAFU | | CUBASS | | CVFATP | |
| | Time 1 | Time 2 | Time 1 | Time 2 | Time 1 | Time 2 |
| Pre- | 15.9 | 16.0 | 15.0 | 14.5 | 19.5 | 17.5 |
| dose | | | | | | |
| 15 min | 17.0 | 17.0 | 14.0 | 13.5 | 23.5 | 22.0 |
| 30 min | 19.0 | 18.4 | 16.0 | 16.0 | over | over |
| | | | | | 120 | 150 |
| 1 hr | over | over | 14.7 | 14.7 | over | over |
| | 100 | 150 | | | 150 | 150 |
| 2 hr | over | over | 16.1 | 15.2 | over | over |
| | 150 | 150 | | | 110 | 150 |
| 4 hr | 15.3 | 15 | over | over | 16.6 | 16.7 |
| | | | 80 | 150 | | |
| 7 hr | 15.5 | 15.5 | over | over | 17.3 | 16.7 |
| | | | 120 | 150 | | |

Dose: 20 mg/kg Fucoidan

TABLE 2
aPTT Assay Results for 5 mg/kg Oral Dosage Amount

| | Dog #1 | | Dog #2 | | Dog #3 | |
|---|---|---|---|---|---|---|
| | CTAAFU | | CUBASS | | CVFATP | |
| | Time 1 | Time 2 | Time 1 | Time 2 | Time 1 | Time 2 |
| Pre- | 15.5 | 14.0 | 18.5 | 18.4 | 16.0 | 16.5 |
| dose | | | | | | |
| 15 min | 20.0 | 22.4 | 18.5 | 19.0 | 21.0 | 20.4 |
| 30 min | 19.9 | 18.0 | 23.0 | 22.0 | 17.9 | 16.5 |
| 1 hr | 17.4 | 17.0 | 19.0 | 20.0 | 19.9 | 19.5 |
| 2 hr | 18.5 | 19.0 | 16.5 | 17.5 | 22 | 22.5 |
| 4 hr | no sample | | 19.7 | 17.4 | 16.5 | 16.0 |
| 7 hr | 16.0. | 14.0 | 13.9 | 16.5 | 17.5 | 16.0 |

Dose: 5 mg/kg Fucoidan

TABLE 3
dPT Assay Results for 20 mg/kg Oral Dosage Amount

| | Dog #1 | | Dog #2 | | Dog #3 | |
|---|---|---|---|---|---|---|
| | CTAAFU | | CUBASS | | CVFATP | |
| | Time 1 | Time 2 | Time 1 | Time 2 | Time 1 | Time 2 |
| Pre- | 42.5 | 45.5 | 46.0 | 51.0 | 48.5 | 52.5 |
| dose | | | | | | |
| 15 min | 43.5 | 45.5 | 40.0 | 44.0 | 66.0 | 71.0 |
| 30 min | 48.5 | 52.0 | 41.0 | 45.0 | 54.5 | 58.0 |
| 1 hr | 56.5 | 55.5 | 39.0 | 42.5 | 49.0 | 52.0 |
| 2 hr | 58.0 | 62.5 | 41.5 | 44.5 | 45.4 | 41.5 |
| 4 hr | 34.0 | 38.5 | 72.0 | 78.0 | 44.5 | 42.4 |
| 7 hr | 38.5 | 41.5 | over | over | 43.7 | 44.3 |
| | | | 150 | 150 | | |

Dose: 20 mg/kg Fucoidan

TABLE 4
dPT Assay Results for 5 mg/kg Oral Dosage Amount

| | Dog #1 | | Dog #2 | | Dog #3 | |
|---|---|---|---|---|---|---|
| | CTAAFU | | CUBASS | | CVFATP | |
| | Time 1 | Time 2 | Time 1 | Time 2 | Time 1 | Time 2 |
| Pre- | 43.5 | 43.0 | 60.0 | 61.9 | 48.5 | 47.5 |
| Dose | | | | | | |
| 15 min | 63.5 | 62.0 | 69.0 | 66.4 | 67.0 | 66.5 |
| 30 min | 63.4 | 63.5 | 76.0 | 77.5 | 52.5 | 50.5 |
| 1 hr | 52.4 | 51.5 | 63.5 | 63.5 | 67.5 | 67.4 |
| 2 hr | 63.5 | 63.0 | 47.0 | 47.0 | 63.5 | 62.5 |
| 4 hr | 43.5 | 43.5 | 37.0 | 37.0 | 43.5 | 41.5 |
| 7 hr | 42.5 | 42.9 | 42.5 | 42.4 | 47.5 | 43.0 |

Dose: 5 mg/kg Fucoidan

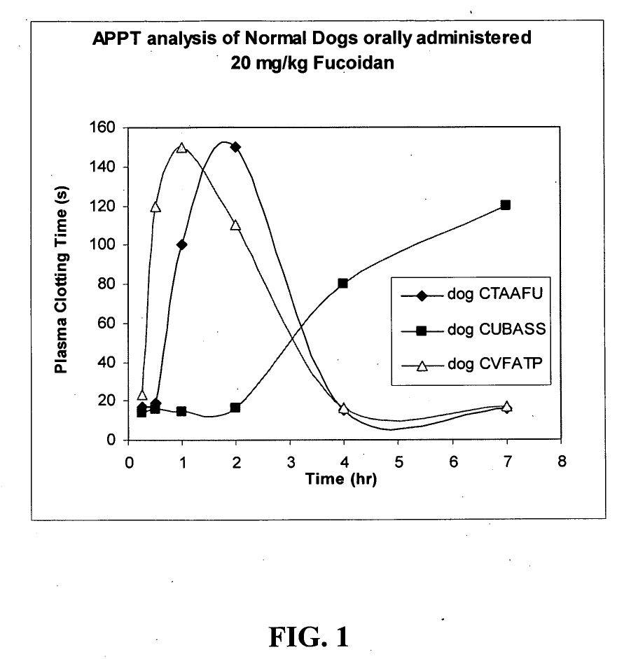

The results of the aPPT analysis for each of the three normal dogs at an oral dose of 20 mg/kg fucoidan are shown in FIG. 1.

The plasma clotting assay results revealed that orally administered fucoidan has the potential to be a safe and effective oral anticoagulant for animal and human use. Specifically, fucoidan administered orally to normal beagle dogs exhibited dose-related anticoagulant activity in each of 3 dogs as detected by substantial (>50%) increases in the APTT, the most commonly utilized clinical measurement for plasma anticoagulation. In looking at the results in Table 1, it can be seen that oral administration of 20 mg/kg of an illustrative fucoidan composition was effective to increase plasma clotting times in each of the three dogs by approximately five to over nine times the pre-dose values within times ranging from about 1 hour to 7 hours post administration. For dogs #1 and #2, significant increases (i.e., from approximately six times to over nine times initial pre-dose values) in plasma clotting times were observed within about 0.5 hours to 1 hour post-administration. Moreover, for dogs #1 and #2, plasma clotting times returned to essentially pre-dose values within several hours post-administration, indicating a lessened propensity for bleeding when compared to a drug such as warfarin, which possesses an extended duration of action that lasts for several days. The Applicants also observed a trend for prolongation at 5 mg/kp p.o.

Oral fucoidan, at the doses tested, did not substantially (>50%) prolong the dilute prothrombin time (dPT) clotting. Without being bound by theory, such results can be interpreted to reflect a limited effect on the “spark” for clotting initiation by the extrinsic pathway.

As stated above, the duration of anticoagulant action for oral fucoidan in normal beagle dogs was several hours, thus reflecting its enhanced safety potential when compared to an anticoagulant such as warfarin.

While the preferred embodiments of the invention have been illustrated and described, it will be appreciated that various changes can be made therein without departing from the spirit and scope of the invention.

What is claimed is:

1. A method for treating a mammalian subject in need of reduced blood coagulation comprising orally administering to said subject a composition comprising a therapeutically effective amount of one or more fucoidans.

2. The method of claim 1, wherein said composition comprises from 5 to 25 percent by weight sulfur.

3. The method of claim 1, wherein said one or more fucoidans are algal.

4. The method of claim 3, wherein said one or more fucoidans are derived from the genus Fucus or Laminaria.

5. The method of claim 4, wherein said one or more fucoidans are derived from Fucus vesiculosis or from Laminaria japonica.

6. The method of claim 1, wherein said administering is effective to produce a greater than 50% increase in said subject's activated partial thromboplastin time (APTT).

7. The method of claim 1, wherein said mammalian subject is an animal.

8. The method of claim 1, wherein said mammalian-subject is a human.

9. The method of claim 1, wherein said composition is administered at a daily dosage ranging from about 1 mg fucoidan/kg to about 50 mg fucoidan/kg.

10. The method of claim 1, wherein said composition is essentially free of algal components other than said one or more fucoidans.

11. The method of claim 1, wherein said administering comprises administering said composition at an initial dosage of from about 1 mg fucoidan/kg to about 50 mg fucoidan/kg, wherein said initial dose is effective to (i) produce a greater than 50% increase in said subject's APTT within one hour of said administering, and (ii) result in a measurable anticoagulant effect in said subject for at least about eight hours post-administration.

12. The method of claim 8, wherein said composition is administered to a human subject at a daily dosage ranging from about 50 mg fucoidan to about 4500 mg fucoidan daily.

13. The method of claim 1, wherein said composition is in a powder, tablet, capsule, suspension, or liquid form.

14. The method of claim 1, wherein the subject possesses a thrombotic disorder.

15. The method of claim 14, wherein said subject possesses a condition selected from deep vein thrombosis, pulmonary embolism, myocardial infarction, disseminated intravascular coagulation, unstable angina, and procoagulation or thrombosis induced by Factor VIIa treatment.

16. The method of claim 1, wherein the cause of the need for reduced blood coagulation is pre- or post surgery or another invasive procedure.

17. A composition for oral administration comprising:
one or more fucoidans, and
a pharmaceutically acceptable excipient; wherein said composition is essentially free of algal components other than said one or more fucoidans and is in a powder, tablet, capsule, suspension, or liquid form.

18. The composition of claim 17, wherein said composition comprises from 5 to 25 percent by weight sulfur.

19. The composition of claim 17, wherein said one or more fucoidans are algal.

20. The composition of claim 19, wherein said one or more fucoidans are derived from the genus Fucus or Laminaria.

21. The composition of claim 20, wherein said one or more fucoidans are derived from Fucus vesiculosis or from Laminaria japonica.

22. A kit comprising:
the composition of claim 17 in packaged form, and instructions for orally administering 1 mg/kg to 50 mg/kg of said one or more fucoidans daily for effecting reduced blood coagulation in a mammalian subject.

23. A blood-contacting medical device coated with one or more fucoidans.
